(19) United States
(12) Patent Application Publication
(10) Pub. No.: US 2017/0224643 A1
(43) Pub. Date: Aug. 10, 2017

(54) METHOD FOR AVOIDING OR INHIBITION OF DYSKINESIA

(71) Applicant: Research Foundation of the City University of New York, New York, NY (US)

(72) Inventors: Andreas H. Kottmann, New York, NY (US); Lauren Malave, New York, NY (US); Heike Rebholz, New York, NY (US); Eitan Friedman, New York, NY (US)

(21) Appl. No.: 15/425,435

(22) Filed: Feb. 6, 2017

Related U.S. Application Data
(60) Provisional application No. 62/291,644, filed on Feb. 5, 2016.
(60) Provisional application No. 62/398,682, filed on Sep. 23, 2016.
(60) Provisional application No. 62/431,662, filed on Dec. 8, 2016.

(51) Int. Cl. A61K 31/198; A61K 31/4436; A61K 31/575; A61K 31/5377

(52) U.S. Cl. CPC A61K 31/198; A61K 31/5377; A61K 31/4436; A61K 31/575

(57) ABSTRACT

Certain neurological disorders are treated with dopamine agonists (e.g. L-Dopa) but these treatments unintentionally cause Levodopa-induced dyskinesias (LIDs) or tardive dyskinesia. An improved treatment is described that treats the patient with both a dopamine agonist and a Smoothened (Smo) agonist or with a dopamine agonist and combination of a Smoothened (Smo) agonist together with Amantadine.

### CROSS-REFERENCE TO RELATED APPLICATIONS

This application claims priority to, and is a non-provisional of, U.S. patent applications Ser. No. 62/291,644 (filed Feb. 5, 2016), 62/398,682 (filed Sep. 23, 2016) and 62/431,662 (Dec. 8, 2016) the entirety of each of which are incorporated herein by reference.

### BACKGROUND OF THE INVENTION

Dopamine substitution therapy or pharmacological stimulation of dopaminergic signaling constitute highly efficacious strategies to ameliorate primary symptoms associated with the loss of dopaminergic neurons and/or other hypo-dopaminergic brain states and remains the “gold standard” in the management of Parkinson's Disease and related disorders. Unfortunately, long-term, chronic dopamine substitution or postsynaptic pharmacological stimulation of dopaminergic signaling leads almost always to the progressive manifestation of involuntary movements (i.e. dyskinesia). Dyskinesia represent a debilitating complication of dopamine substitution therapy by levodopa (L-Dopa) and other dopamine substitution therapies like dopamine receptor agonists for Parkinson's Disease (PD). Since L-Dopa is the most efficacious treatment to relieve bradykinesia and akinesia, muscle rigidity and resting tremor in PD, L-Dopa induced dyskinesia (LID) are ultimately experienced by the vast majority of patients. In addition, psychiatric conditions often manifesting as compulsive behaviors, are emerging as a serious problem in the management of L-Dopa therapy. Likewise, the long-term therapeutic stimulation of postsynaptic dopaminergic signaling in many psychiatric conditions also often leads to the development of dyskinesia, often most prominently manifesting as oral-facial dyskinesia. Because of these complications associated with the otherwise highly beneficial and efficacious strategy of augmenting dopaminergic signaling in PD and related disorders or psychiatric indications, enormous efforts have been made to find adjuvant strategies that prevent dyskinesia formation without curtailing the beneficial effects of dopamine substitution therapy. The discussion above is merely provided for general background information and is not intended to be used as an aid in determining the scope of the claimed subject matter.

### BRIEF DESCRIPTION OF THE INVENTION

Certain neurological disorders are treated by dopamine substitution (e.g. L-Dopa,) or agents that simulate dopamine dependent signaling (e.g dopamine receptor agonists) but these treatments unintentionally cause debilitating dyskinesia known in the field as Levodopa-induced dyskinesia (LIDs) and tardive dyskinesia. An improved treatment is described that treats the patient with either L-Dopa or a dopamine agonist together with an agonist of the G-coupled receptor Smoothened (Smo), which is the receptor for the natural ligand Sonic Hedgehog (Shh). Importantly, applicant finds that the application of Smo agonists inhibits the formation and expression of dyskinesia caused by dopamine substitution in well established pre-clinical models of dyskinesia.

In a first embodiment, a method for treating Parkinson's Disease is provided. The method comprising administering to a patient experiencing Parkinson's Disease both (1) a dopamine agonist and (2) a smoothened (Smo) agonist.

In a second embodiment, a method for treating dyskinesia is provided. The method comprising steps of identifying a patient as having dyskinesia; administering a dopamine agonist to the patient; administering a smoothened (Smo) agonist to the patient; repeating the step of administering the dopamine agonists and the step of administering the smoothened (Smo) agonist, the repeating occurring at a predetermined frequency.

In a third embodiment, a method for treating dyskinesia, the method comprising administering to a patient experiencing dyskinesia both amantadine and a smoothened (Smo) agonist.

This brief description of the invention is intended only to provide a brief overview of subject matter disclosed herein according to one or more illustrative embodiments, and does not serve as a guide to interpreting the claims or to define or limit the scope of the invention, which is defined only by the appended claims. This brief description is provided to introduce an illustrative selection of concepts in a simplified form that are further described below in the detailed description. This brief description is not intended to identify key features or essential features of the claimed subject matter, nor is it intended to be used as an aid in determining the scope of the claimed subject matter. The claimed subject matter is not limited to implementations that solve any or all disadvantages noted in the background.

### BRIEF DESCRIPTION OF THE DRAWINGS

So that the manner in which the features of the invention can be understood, a detailed description of the invention may be had by reference to certain embodiments, some of which are illustrated in the accompanying drawings. It is to be noted, however, that the drawings illustrate only certain embodiments of this invention and are therefore not to be considered limiting of its scope, for the scope of the invention encompasses other equally effective embodiments. The drawings are not necessarily to scale, emphasis generally being placed upon illustrating the features of certain embodiments of the invention. Thus, for further understanding of the invention, reference can be made to the following detailed description, read in connection with the drawings in which:

As well established in the field of Parkinson's Disease treatment and—research, in this disclosure “L-Dopa” references a solution in 0.9% saline of L-Dopa together with benserazide, an inhibitor of L-Dopa break-down. If the amount of L-Dopa that is injected is 5 mg/kg, then the benserazide dose is 10 mg/kg. If the amount of L-Dopa that is injected is 10 to 30 mg/kg, then the benserazide dose is 20 mg/kg.

FIG. 1 depicts assessment of 6-OHDA induced dopaminergic lesion. All animals of cohorts A and B were lesioned unilaterally by stereotactic injection of 6-OHDA into the dorso-lateral striatum. White bars: saline injected, black bars: L-dopa (5 mg/kg, IP) injected. Unilateral striatal 6-OHDA injection results in ipsilateral dopamine hypersensitivity. (**=p<0.001, ***=p<0.0001, t-test; n=9-11/treatment group; mean±SEM);

FIG. 2 shows that a single dose of cyclopamine (5 mg/kg; IP; an antagonist of Smoothened) increases by 60% “abnormal induced movements” (AIMs, i.e. dyskinesia) that were induced by daily dosing with L-Dopa (5 mg/kg; IP) for 14 days. (**=p<0.001, t-test; n=7-8/treatment group; mean±SEM);

FIG. 3 depicts quantification of dyskinisia in the aphakia mouse line, a genetic model of LID. Dosing with cyclopamine (5 mg/kg; IP; an antagonist of the receptor smoothened) increases abnormal induced movements (AIMs) that were induced by daily dosing with L-Dopa (20 mg/kg; IP). (*=p<0.01, **=p<0.001, ***=p<0.0001, t-test; n=9-11/treatment group; mean±SEM);

FIG. 4 shows quantification of progressive induction of LID by daily injections of L-Dopa (10 mg/kg; IP) in mice with genetic ablation of Shh expression from dopamine neurons. LID form in mutants (black bars) but not in litter controls (white bars). (*=p<0.01, t-test; n=4-5/treatment group and genotype; mean±SEM);

FIG. 5 depicts that a single dose of SAG (10 mg/kg; IP, an agonist of the receptor smoothened) leads to a 2 fold reduction of abnormal induced movements (AIMs) that progressively formed by daily dosing with L-Dopa (5 mg/kg; IP) over 16 days. (*=p<0.01, t-test; n=5-11/treatment group; mean±SEM);

FIG. 6 depicts that a single dose of Purmorphamine (15 mg/kg; IP ; PUR, an agonist of the receptor smoothened) co-injected with L-Dopa (20 mg/kg, IP) leads to a 2 fold reduction of abnormal induced movements (AIMs) that progressively formed by daily dosing with L-Dopa (5 mg/kg; IP) over 16 days. (*=p<0.01, t-test; n=5-11/treatment group; mean±SEM);

FIG. 7 shows that chronic dosing with SAG together with L-Dopa (as an “adjuvant” strategy to L-Dopa) results in a reversible, dose dependent build-up of a reduction of abnormal induced movements (AIMs) in the unilateral 6-OHDA model of dyskinesia. Chronic drug doses, wash out periods, and within subject drug escalations indicated in figure. (*=p<0.01, **=p<0.001, t-test; n=7-12/treatment group; mean±SEM);

FIG. 8 depicts dose titration with SAG together with L-Dopa (as an “adjuvant” strategy to L-Dopa) and reveals a build-up of a reduction of abnormal induced movements over 7 days (AIMs) using the unilateral 6-OHDA model of dyskinesia. The kinetics of the build-up is SAG dose dependent. (*=p<0.01, t-test; n=6-9/treatment group; mean±SEM);

FIG. 9 depicts that dosing with SAG (an agonist of the receptor smoothened) decreases abnormal induced movements (AIMs) that were induced by daily dosing with L-Dopa in the aphakia mouse model of LID. (**=p<0.001, t-test; n=7-8/treatment; mean±SEM);

FIG. 10 shows that the expression of dyskinesia observed in mice with genetic ablation of Shh from dopamine neurons is dose dependently inhibited by SAG treatment. 3 paw dyskinesia induced by daily injections of L-Dopa (25 mg/kg; IP) over 20 days (black columns labeled baseline, BL, d6, d12, d20, were reduced more than 2 fold by a single injection of SAG (10 mg/kg; IP) on day 21, and about 10 fold by a single injection of SAG (20 mg/kg; IP) on day 22 in miceLlD expressed as a ratiometric value of dyskinesia observed in mutants over controls. (*=p<0.01, **=p<0.001, t-test; n=10/treatment/genotype group; mean±SEM);

FIG. 11 depicts a summary of all dyskinesia measures across two models of dyskinesia and three structurally different compounds (Cyclopamine [antagonist], SAG [agonist], Purmorphamine [Pur, agonist]) of the receptor Smoothened. Abnormal induced movements (AIMs) are expressed as ratio over untreated controls. Dyskinesia scores in controls are set to 1 (dashed line). Cyclopamine, (a, antagonist), increases dyskinesia while SAG (b,c, agonist) or Purmorphamine, (d, agonist), decrease dyskinesia. (*=p<0.01, **=p<0.001,* **=p<0.0001, t-test; n=28-32/treatment group; mean±SEM);

FIG. 12 depicts a synergistic, 5 fold inhibition of L-Dopa induced dyskinesia by SAG in combination with amantadine (a well-established antagonist of glutamate signaling and used as a treatment for dyskinesia) in the Aphakia mouse model of dyskinesia. (**=p<0.001, t-test; n=5-13/treatment; mean±SEM).

### DETAILED DESCRIPTION OF THE INVENTION

Disclosed in this specification is a method to find an adjuvant strategy that blocks the unwanted side effects of dopamine substitution and dopamine receptor stimulation. The method is based on the understanding that, in disease, not only dopamine (DA) production and/or signaling is reduced but also the function of as of yet unidentified other factor(s). DA substitution, in absence of also reverting those other factor(s) to their normal effective function and concentration, results in unwanted side effects. Beneficial treatment methods can be identified by (1) identifying appropriate pharmacology that allows normalization of the function of the effected, additional signal transduction pathways; (2) demonstrating that co-administration of L-Dopa together with compounds that normalize the effected, additional signal transduction pathways block the formation and/or the expression of Levodopa-induced dyskinesias (LIDs).

Dopamine (DA) neurons utilize multiple means by which they communicate with their targets. The release of all of these factors, in addition to DA, should be diminished in Parkinson Disease (PD) patients due to the progressive loss of DA neurons. Selective augmentation of DA signaling without concomitantly increasing other dopamine-cell specific cell signaling may contribute to L-Dopa induced dyskinesia, LID.

Without wishing to be bound to any particular theory, applicant believes that (1) in PD (which is characterized by a progressive degeneration of DA neurons which produce both, dopamine and Sonic Hedgehog (Shh)) DA levels and Shh levels drop; (2) a pharmacological compensation of the loss of DA neurons should attempt to normalize both DA and Shh signaling; (3) DA substitution by only L-Dopa results in LIDs because of reduced Shh signaling causing an imbalance between dopamine and Shh signaling (4) LID formation and expression by L-Dopa can be suppressed by adjuvant treatment with agonists of the receptor Smoothened (Smo) resulting in increased Shh signaling.

This disclosure demonstrates that, in the well-established unilateral 6-OHDA model of L-Dopa induced dyskinesia and in the well-established genetic model of L-Dopa induced dyskinesia, the “aphakia” mouse line: (1) Pharmacological stimulation of Shh signaling by purmorphamine (a Smo agonist) results in delayed and weaker/blunted induction and reduced expression of dyskinesia compared to controls; (2) Pharmacological stimulation of Shh signaling by SAG (a Smo agonist) results in delayed and weaker/blunted induction and reduced expression of dyskinesia compared to controls; (3) Pharmacological inhibition of Shh signaling by cyclopamine (a Smo antagonist) results in more rapid induction and stronger expression of dyskinesia compared to controls. (4) the drug combination of SAG (a Smo agonist) with Amantadine (a glutamate antagonist), results in a synergistic, 5 fold reduction in the expression of dyskinesia.

The clinical significance of these results is that the pharmacological stimulation of Smo signaling as an adjuvant treatment to dopamine receptor agonist-treatment (e.g. L-Dopa treatment) might extend the utility of dopamine substitution or dopamine receptor stimulation (resp.) therapy in PD by inhibiting LID formation and expression. For example in Parkinson's Disease (PD), Shh as well as DA levels must diminish due to progressive DA neuron degeneration. However, only DA in form of L-Dopa or dopaminergic agonists, but not Shh, is supplemented by current pharmacological PD management regimes. Secondary pathologies like dyskinesias, in particular L-Dopa induced dyskinesia (LID) or tardive dyskinesia (often induced by dopamine agonists or certain dopamine receptor antagonists) that are associated with long term supplementation of dopamine or alterations of dopaminergic signaling manifests, in part, because of an absence of concomitant Shh mediated signaling. Consistent with this hypothesis, applicant finds that inhibition of G-coupled receptor Smoothened (Smo, the critical mediator of Shh signaling) is a strong facilitator of LID formation and expression in the standard models of LID in mice. Conversely, the pharmacological stimulation of Smo by its agonists, Purmorphamine or SAG, inhibits the formation and expression of LID. In addition, applicant finds that Amantadine, an unrelated and well-characterized compound that regulates an unrelated pathway, namely glutamatergic signaling and is known to reduce LID in Parkinson's Disease patients, synergizes with SAG in two different models of LIDs leading to a 5 fold reduction in dyskinesia. Applicant's results therefore suggest that Smo agonist-adjuvant treatment by itself or in combination with amantadine might reduce LID formation and expression in Parkinson's Disease and other diseases in which LID or tardive dyskinesia form.

Additional Shh agonists contemplated for use with this disclosed method include the Shh agonists, “SAG 1.3” (CAS No. 364590-63-6); “20(S)—OHC” (CAS No. 516-72-3); “20(S)-yne” (CAS No. 1397692-46-4); “7-Keto-27-OHC.” “22(s)-OHC”; 20 “(S)—OHC-Bu-n-butyl”; “20(S)—OHC-Pent-n-pentyl”; “20(S)—OHC-PentSat”; “GSA-10”.

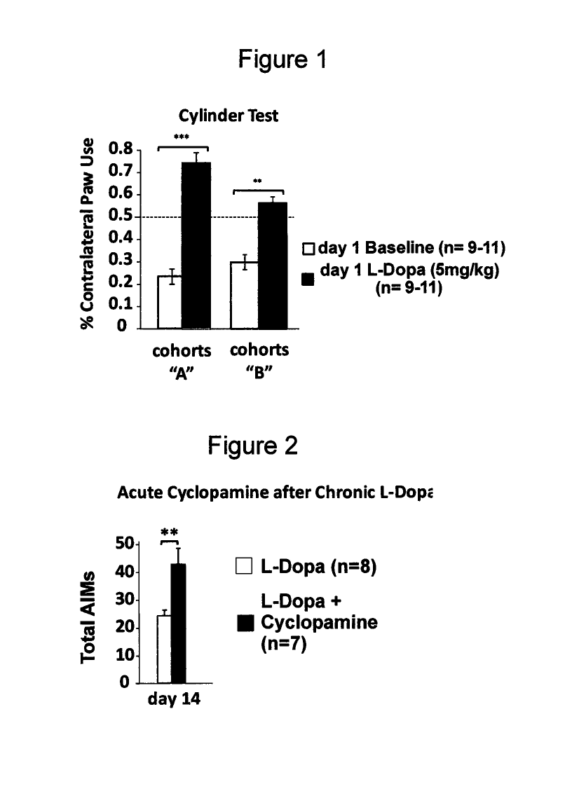

Assessing dopamine hyper-sensitivity in the unilateral 6-OHDA model of LID. The well-established unilateral 6-OHDA model of LIDs was used in which the injection of the neurotoxin results in unilateral DA hypersensitivity due to a greater than 80% DA neuron degeneration, and subsequent dopamine hypersensitivity. Two weeks after 6-OHDA injection, the degree of functional deficits are quantified by assessing contralateral paw use in a “cylinder test” (FIG. 1). In untreated, 6-OHDA injected animals contralateral paw use during rearing at the glass wall is reduced from random (50%, dotted line) due to the dopaminergic lesion and resulting reduced dopamine secretion (FIG. 1, white bars). Upon dopamine substitution by a single dose of L-Dopa contralateral paw use is increased above random (50% dotted line) due to dopamine hypersensitivity that developed in response to reduced dopamine levels (FIG. 1 black bars).

Inhibition of Smo by cyclopamine INCREASES LIDs After the functional assessment of dopaminergic denervation in the 6-OHDA model of LIDS, LIDs are induced by daily injections of L-Dopa (5 mg/kg, IP) for 13 days followed by quantification of dyskinesia. Abnormal movements are assessed for 1 min every 20 min, over a period of 120 min immediately after L-DOPA administration coinciding with the peak of L-Dopa dependent DA release. Abnormal induced movements (AIMs), clearly distinct from natural stereotyped behaviors (i.e., grooming, sniffing, rearing, and gnawing), are classified following a well-established scheme in the field of dyskinesia research into four different subtypes: locomotive (tight contralateral turns), axial (contralateral dystonic posture of the neck and upper body), limb (jerky and fluttering movements of the limb contralateral to the side of the lesion), and orofacial (vacuous jaw movements and tongue protrusions) (Sebastianutto, I., N. Maslava, C. R. Hopkins, and M. A. Cenci, Validation of an improved scale for rating l-DOPA-induced dyskinesia in the mouse and effects of specific dopamine receptor antagonists. Neurobiology of disease, 2016. 96: p. 156-170.). The co-administration of the Smo antagonist cyclopamine (5 mg/kg, IP) together with L-Dopa (5mg/kg, IP) at day 14 results in an approximately 80% increase in LIDs (FIG. 2, black bar) compared to L-Dopa alone (FIG. 2, white bar).

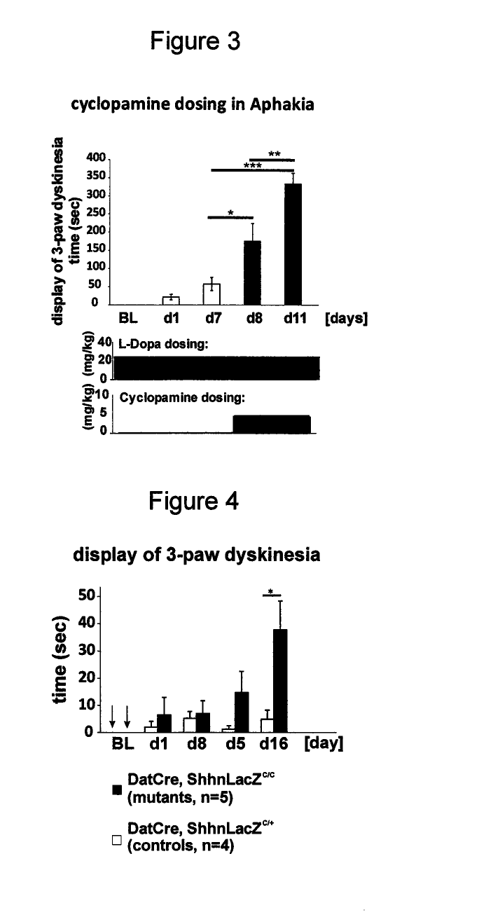

Inhibition of Smo by cyclopamine in thegenetic “aphakia ” model of dyskinesia INCREASES LIDs. Analogous to the use of the unilateral 6-OHDA model of LID, we tested whether Shh signaling effects the formation and display of LID in an independent, genetic model, the well-studied Aphakia mouse model. This mouse line reveals severely reduced dopaminergic innervation of the striatum leading to a chronic dopamine hypersensitive brain. The hypo-dopaminergic state of these animals is thought to be similar to the dopaminergic state of Parkinson's disease patients. It is well established that these mice readily develop LID upon semi chronic dosing with L-Dopa. Guided by Ding et al, 2011 (Ding, Y., L. Won, J. P. Britt, S. A. Lim, D. S. McGehee, and U. J. Kang, Enhanced striatal cholinergic neuronal activity mediates L-DOPA-induced dyskinesia in parkinsonian mice. Proceedings of the National Academy of Sciences of the United States of America, 2011. 108(2): p. 840-5.), who found that “three paw dyskinesia”, recognized as abnormal sliding movements upon rearing in a glass cylinder, peaks at 15′ after L-dopa injection into aphakia mice, we scored dyskinetic movements from video footage taken for 15 minutes beginning 10′ after L-Dopa injection. Daily systemic injections of L-Dopa (20 mg/kg, IP) results in progressive display of 3 paw dyskinesia over 7 days (FIG. 3, white bars. Co-injection of cyclopamine (5 mg/kg, IP) together with L-Dopa (20 mg/kg, IP) on day 8 results in a roughly 3 fold increase in 3-paw dyskinesia compared to day 7 (FIG. 3, see d8, black bar). Further daily co-injections of cyclopamine together with L-Dopa (5 mg/kg, IP and 20 mg/kg, IP, resp.) results in further increases in dyskinesia scores reaching 7 fold the score obtained after 7 days of L-Dopa only dosing (FIG. 3, see d11, black bar compared to d7, white bar).

Mice with conditional, partial, genetic ablation of Shh from DA neurons develop LIDs upon semi chronic dosing with L-Dopa: In Shh-nLacZ^{C/C}/Dat-Cre mice Shh expression in DA neurons is genetically, partially ablated (Gonzalez-Reyes, L. E., M. Verbitsky, J. Blesa, V. Jackson-Lewis, D. Paredes, K. Tillack, S. Phani, E. R. Kramer, S. Przedborski, and A. H. Kottmann, Sonic hedgehog maintains cellular and neurochemical homeostasis in the adult nigrostriatal circuit. Neuron, 2012. 75(2): p. 306-19). These mice, presumably, have reduced levels of Shh in the brain and therefore, presumably, reduced levels of Smo activation. Consistent with the pharmacological inhibition of Smo in the 6-OHDA and aphakia models of LID, applicant finds that daily dosing with L-Dopa (10 mg/kg;IP)) results in formation of LID over time (i.e. 16 days) in Shh-nLacZ^{C/C}/Dat-Cre mice (FIG. 4, black bars) but not in litter controls (i.e. in animals with normal expression of Shh and, presumably, normal Smo activation) (FIG. 4 white bars).

In summary these experiments revealed that (1) the pharmacological inhibition of Smo in two well -established models of LID (unilateral 6OHDA model and aphakia mouse line model) or (2) the genetic ablation of the natural activator of Smo, Shh, in mice, results in INCREASED LID formation and expression.

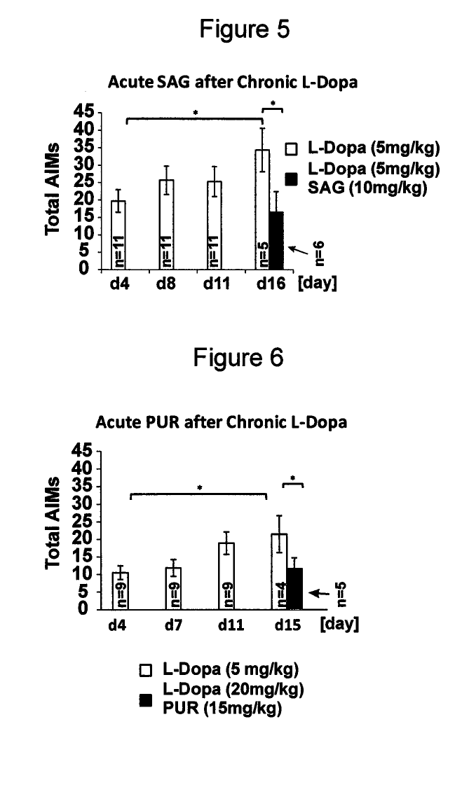

Stimulation of Smo by SAG DECREASES LIDs. Analogous to the experiments testing the inhibition of Smo, the unilateral 6-OHDA model of LIDs was used to test the effect of activating Smo signaling by the agonist SAG (FIG. 5). Two weeks after 6-OHDA injection, LIDs are induced by daily injections of L-Dopa (5 mg/kg, IP) for 16 days. A gradual increase in the display of dyskinesia was observed from day 4 to day 16 (FIG. 5, white bars). Consistent with the hypothesis and conversely to the facilitation of LIDs by the inhibition of Smo signaling, applicant found about a twofold REDUCTION in the display of LIDs upon the co-administration of a L-Dopa dose (5 mg/kg, IP) together with the Smo Agonist SAG (10 mg/kg, IP) on day 16 (FIG. 5, black bar).

Stimulation of Smo by Purmorphamine DECREASES LIDs. Analogous to the experiments testing the inhibition of Smo, the unilateral 6-OHDA model of LIDs was used to test the effect of activating Smo signaling by the agonist Purmorphamine (PUR). Two weeks after 6-OHDA injection, LIDs are induced by daily injections of L-Dopa (5 mg/kg, IP) for 14 days. A gradual increase in the display of dyskinesia was observed from day 4 to day 11 (FIG. 6, white bars). Consistent with the hypothesis and similar to the results with stimulating Smo with SAG and conversely to the facilitation of LIDs by the inhibition of Smo signaling, applicant finds about 40% REDUCTION in the display of LIDs upon the co-administration of a L-Dopa “challenge” dose (20 mg/kg; IP, i.e. 4×the regular dose) together with the Smo agonist Purmorphamine (15 mg/kg, IP) on day 15 (FIG. 6, black bar).

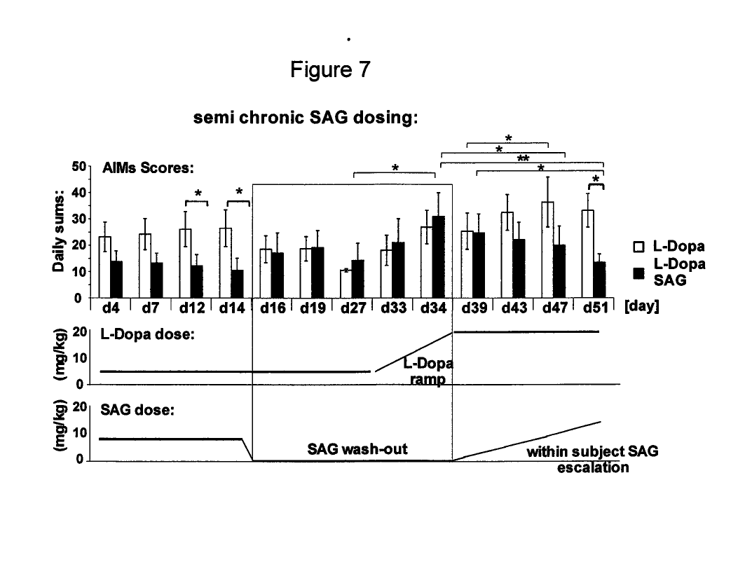

Chronic dosing with SAG together with L-Dopa as an adjuvant strategy to L-Dopa reveals a reversible, dose dependent build-up of a REDUCTION of dyskinesia using a within subject drug escalation scheme: Analogous to the experiments testing the acute stimulation of Smo, the unilateral 6-OHDA model of LIDs was used to test the effect of semi chronic activation of Smo signaling by the agonist SAG. 2 weeks after 6-OHDA injection animals were either daily dosed with L-dopa (5 mg/kg, IP) alone or with L-dopa (5 mg/kg, IP) together SAG (10 mg/kg, IP) together for 14 days. Dyskinesia (AIMS) scores increased progressively in the L-Dopa only group (FIG. 7 white bars d4 to d14) while AIMS scores progressively declined in the L-Dopa/SAG group leading to a greater than 2 fold reduction over the control group on day 14 (FIG. 7: black bars d4 to d14). Animals were maintained on L-Dopa (5 mg/kg, IP, once daily injections) without SAG for the next 13 days to test whether dyskinesia would return in those mice that had received L-Dopa together with SAG (FIGS. 7: d16 to d34; labeled “SAG wash-out”). Absence of SAG led to an immediate reversal of LID scores that became indistinguishable from control groups (FIG. 7: white and black bars d16, d19, d27, d33, d34). Beginning at day 33 (d33) daily L-dopa dosage was ramped up to 20 mg/kg, IP over 6 days leading to a 3 fold increase in LID scores in both groups of animals on day 34 compared to day 27 (FIG. 7: black and white bars d27, d33, d34.) Applicant then used within subject drug escalation of SAG from 1.5 mg/kg, IP at day 39.5 mg/kg, IP at day 43, 10 mg/kg, IP at day 47, to 15 mg/kg, IP at day 51 (FIG. 7 black bars) and found a dose dependent and greater than 2 fold reduction in LIDs with 15 mg/kg of SAG compared to controls on day 51 (FIG. 7: d39, d43, d47, d51 white bars).

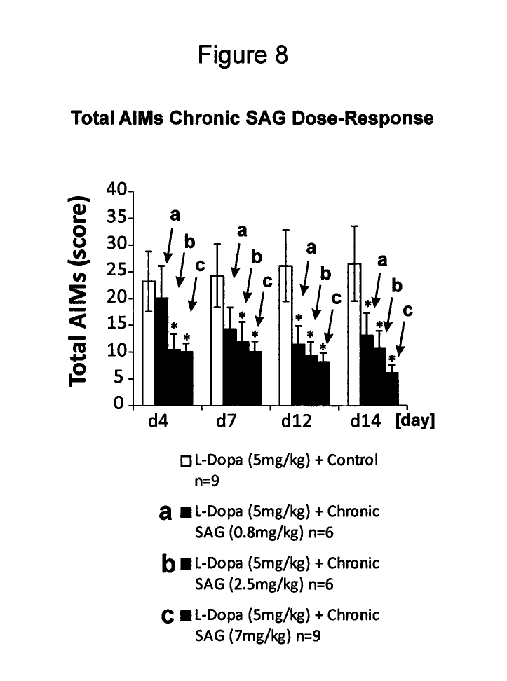

Chronic dosing with SAG together with L-Dopa as an adjuvant strategy to L-Dopa reveals a reversible, dose dependent build-up of a REDUCTION of dyskinesia using separate (drug naïve) groups for each SAG concentration. Analogous to the experiments testing the dose dependent effects of chronic stimulation of Smo in a within subject paradigm, the unilateral 6-OHDA model of LIDs was used to test the dose dependent effect of semi chronic activation of Smo signaling by the agonist SAG in groups of drug nave animals. 2 weeks after 6-OHDA injection animals were either daily dosed with L-dopa (5 mg/kg, IP) alone (FIG. 8, white bars) or with L-dopa (5 mg/kg, IP) together with SAG, IP at 0.8, or 2.5, or 7 mg/kg (FIG. 8, labeled a, b, c resp.) for 14 days. The lowest concentration of SAG (0.8 mg/kg, IP, FIG. 8 black bars labeled “a”) led to a significant reduction of dyskinesia at day 12 (45% reduction) of daily dosing, while daily dosing with 2.5 mg/kg SAG results in 50% reduction of dyskinesia already at 4 days of dosing. The highest concentration of SAG (7 mg/kg, IP) leads to a progressive reductions in dyskinesia from about 50% at day 4 to a about 80% reduction at day 14 (FIG. 8, white bars: controls, black bars labeled “c”:).

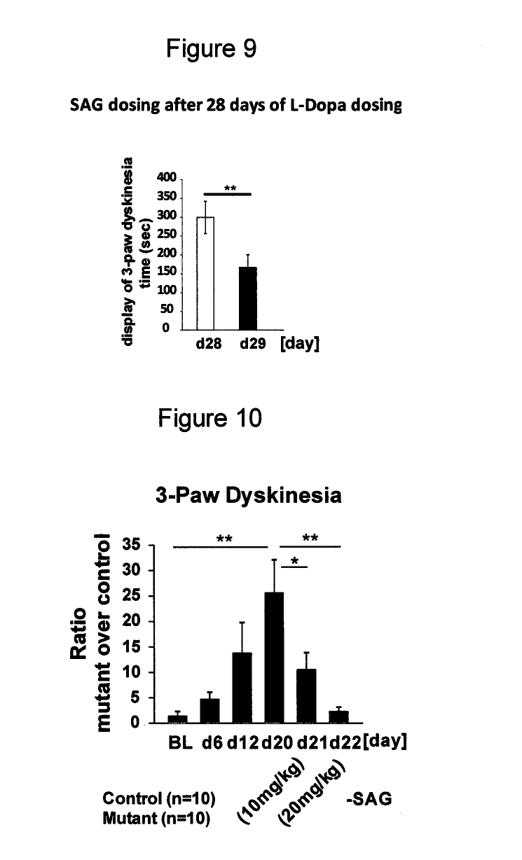

Stimulation of Smo by SAG in a genetic model of dyskinesia DECREASES LIDs. Analogous to testing the effect of cyclopamine, we used the aphakia mouse line to determine whether stimulation of Shh signaling might reduce the display of established LID. We injected daily for 28 days L-Dopa (20 mg/kg; IP). On day 29 we co-injected SAG (20 mg/kg, IP) together with L-Dopa (20 mg/kg, IP). We scored 3 paw dyskinesia at day 28 (FIG. 9: white bar) and day 29 (FIG. 9: black bar) and find a roughly 2 fold reduction in dyskinesia upon SAG dosing.

Acute injection of SAG into mice with conditional, partial, genetic ablation of Shh from DA neurons reduces the display of L-Dopa induced dyskinesia. As before, applicant finds that daily dosing with L-Dopa (25 mg/kg, IP) over a course of 20 days results in gradual, robust formation of LID in Shh-nLacZ^{C/C}/Dat-Cre mice (FIG. 10: BL (baseline) to d 20; dyskinesia expressed as ratio of dyskinesia measured in mutant mice over litter controls) but not in litter controls. Using a “within subject” drug escalation regiment, applicant finds that co-injection of SAG (10 mg/kg, IP) together with L-Dopa (25 mg/kg; IP) on day 21 reduces the dyskinesia score 2.5 fold compared to day 20. Co-injection of SAG (20 mg/kg, IP) together with L-Dopa (20 mg/kg, IP) on day 22 reduces dyskinesia scores about 10 fold compared to day 20 (FIG. 10, columns labeled d20, d21, d22, resp.). These results indicate that LID in mice with reduced Shh signaling can be strongly reduced by pharmacological stimulation of Smo.

In summary these results revealed that the acute or chronic pharmacological stimulation of Smo in two well -established models of LID (unilateral 6-OHDA model and aphakia mouse line model) or in mice with genetic ablation of the natural activator of Smo, Shh, results in profound inhibition of LID formation and expression.

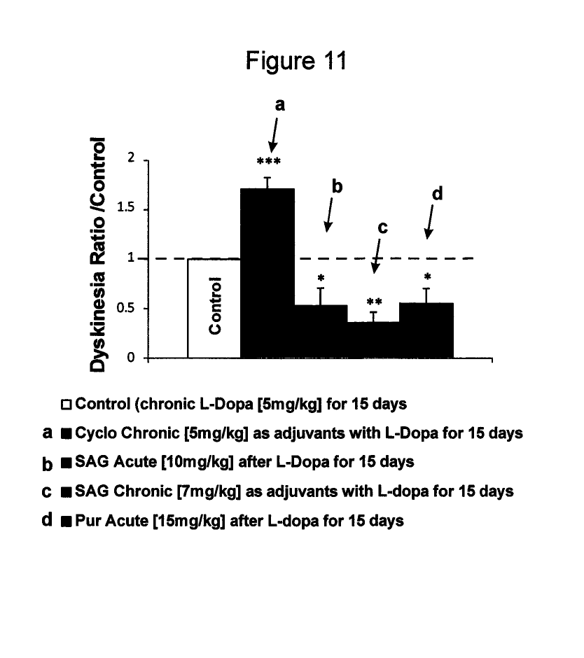

Meta-analysis of dyskinesia measurements across two models of LID and three structurally distinct compounds (cyclopamine, SAG, purmorphamine):. A ratiometric expression of LID after treatment with Smo antagonist or agonists over L-Dopa only treated controls allows applicant to present results combined across two distinct models of dyskinesia and three distinct compounds. Setting dyskinesia scores observed in L-Dopa only treated controls as 1 (FIG. 11: dotted line and white bar), we find that inhibition of Smo by cyclopamine results in a 70% INCREASE of dyskinesia (FIG. 11: black bar labeled “a”). Conversely, acute stimulation of Smo by SAG leads to a roughly 2 fold REDUCTION in the expression of established dyskinesia (FIG. 11, black bar labeled “b”) and semi chronic daily dosing with SAG together with L-Dopa (“adjuvants” strategy) leads to about 70% reduction in the formation and display of LIDs (FIG. 11, black bar labeled “c” Consistent and similar to the effect of SAGthe acute stimulation of Smo with Purmorphamine leads to a roughly 2 fold REDUCTION in the display of established LID (FIG. 11, black bar labeled “d”).

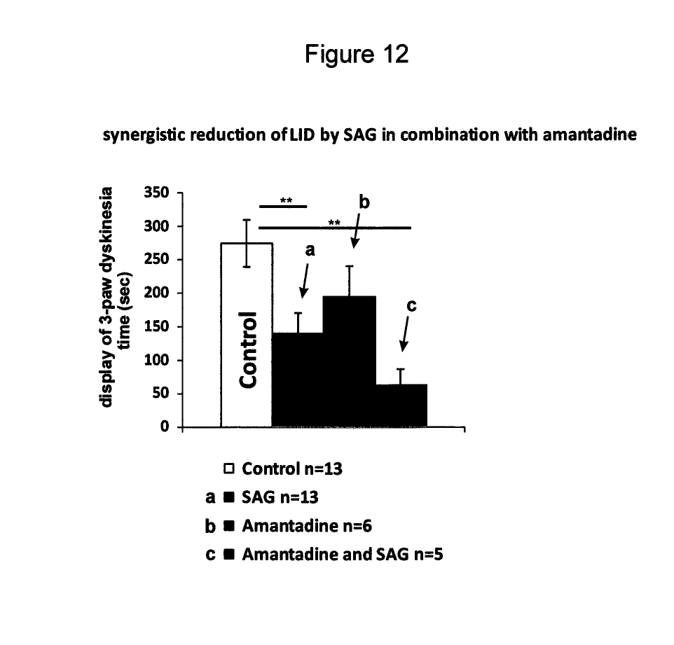

Stimulation of Smo combined with Amantadine, a FDA approved compound for the treatment of LID results in SYNERGISTIC INHIBITION of LIDs, Amantadine (CAS 768-94-5) is a gluatamate antagonist and leads to a 20 to 30% inhibition of dyskinesia in animal models (Sebastianutto, I., N. Maslava, C. R. Hopkins, and M. A. Cenci, Validation of an improved scale for rating l-DOPA-induced dyskinesia in the mouse and effects of specific dopamine receptor antagonists. Neurobiology of disease, 2016. 96: p. 156-170.) and a 23% inhibition of dyskinesia in Parkinson's Patients (Pahwa, R., C. M. Tanner, R. A. Hauser, K. Sethi, S. Isaacson, D. Truong, L. Struck, A. E. Ruby, N. L. McClure, G. T. Went, and M. J. Stempien, Amantadine extended release for levodopa-induced dyskinesia in Parkinson's disease (EASED Study). Movement disorders: official journal of the Movement Disorder Society, 2015. 30(6): p. 788-95.). Analogous to testing the effects of the stimulation of Smo by SAG in the aphakia mouse model of dyskinesia described above we injected daily L-Dopa (25 mg/kg, IP) for 21 days. All animals developed robust dyskinesia scores. On day 21 animals were randomly assigned into 4 groups. The control group received L-Dopa (25 mg/kg, IP) while group A received SAG (20 mg/kg, IP) together with L-Dopa (25 mg/kg, IP), group B received amantadine (40 mg/kg, IP) together with L-Dopa (25 mg/kg, IP); and group C received a combination of SAG and Amantadine (20 mg/kg and 40 mg/kg, resp., IP and L-Dopa (25 mg/kg, IP). 3-paw dyskinesia was quantified. SAG dosing resulted in a 2 fold reduction in the display of LID (FIG. 12: black column labeled “a” compared to controls, white column), Amantadine dosing resulted in a roughly 30% reduction of LID (as observed in the field of dyskinesia research before and similar to the effect size of amantadine in Parkinson's disease patients) that did not reach significance FIG. 10, black column labeled “b” and the combination of SAG together with Amantadine resulted in a 5 fold reduction in LIDs (FIG. 10, black column labeled “c”).

In summary, this disclosure reveals that G protein coupled receptor Smoothened (Smo) mediated Shh signaling is a significant regulator of LID formation and expression. Importantly, applicant's results demonstrate that the manipulation of Smo activity has a dose dependent and treatment duration dependent effect on LID formation and expression such that inhibition of Smo activity results in facilitation of LID formation and expression while stimulation of Smo activity results in reduced LID formation and expression. In particular, the results provide proof of principle that (1) the acute pharmacological stimulation of Smo ameliorates the expression of established LIDs in two standard, well-established preclinical animal models of LIDs, (2) the semi chronic pharmacological stimulation of Smo combined with each dose of L-Dopa reduces formation of dyskinesia leading to a greater decrease in expression of LIDs than achieved with acute dosing, (3) the drug combination of amantadine, the only FDA approved drug for the treatment of LIDs together with SAG (i.e. stimulation of Smo) results in a synergistic, 5 fold reduction in LIDs, that is a far greater reduction in LIDs than achieved by amantadine or SAG alone and (4) inhibiting formation of LID through multiple, repeated treatments with Smo agonist requires 4 to 10 fold smaller doses of agonist than inhibiting expression of established LID through a single, acute dose of agonist. In one embodiment, the multiple, repeated treatments may be spaced from the treatment with the dopamine agonist in time by, for example, less than three hours.

Applicant comes to these conclusions by using (1) two well established technical approaches for scoring dyskinesia, (2) two well established animal paradigms for investigations of dyskinesia, (3) four means of manipulating Smo activity and (4) testing the short as well as long term effects of altering Smo activity. In particular, applicant finds that the reduction of expression of the gene Sonic Hedgehog (Shh), the natural activator of Smo in vivo, or the treatment of dyskinetic mice with the antagonist “Cyclopamine” of Smo leads to a facilitation and/or increase of LID formation and expression. In contrast chronic as well as acute treatment of dyskinetic mice with the agonist “SAG” of Smo, or the agonist “Purmorphamine” of Smo results in profoundly reduced expression of LIDs. Since all of these factors, i.e. Shh, cyclopamine, SAG, and Purmorphamine regulate specifically the activity of the receptor protein Smo, applicant believes that inhibition of Smo activity by any means will lead to facilitation of LID while stimulation of Smo activity by any means will inhibit formation and expression of LID.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

What is claimed is:

1. A method for treating Parkinson's Disease, the method comprising administering to a patient experiencing Parkinson's Disease both (1) a dopamine agonist and (2) a smoothened (Smo) agonist.

2. The method as recited in claim 1, wherein the dopamine agonist is L-dopa.

3. The method as recited in claim 2, wherein the smoothened (Smo) agonist is purmorphamine (PUR).

4. The method as recited in claim 2, wherein the smoothened (Smo) agonist is a smoothened agonist (SAG).

5. The method as recited in claim 2, wherein the smoothened (Smo) agonist is SAG1.5.

6. The method as recited in claim 2, wherein the smoothened (Smo) agonist is 20(S)-OHC.

7. The method as recited in claim 2, wherein the smoothened (Smo) agonist is 20(S)-yne.

8. The method as recited in claim 2, wherein the smoothened (Smo)) agonist is 7-Keto-27-OHC.

9. A method for treating dyskinesia, the method comprising steps of identifying a patient for dyskinesia treatment;
administering a dopamine agonist to the patient;
administering a smoothened (Smo) agonist to the patient;
repeating the step of administering the dopamine agonists and the step of administering the smoothened (Smo) agonist, the repeating occurring at a predetermined frequency.

10. The method as recited in claim 9, wherein the step of administering the dopamine agonist and the step of administering the smoothened (Smo) agonist occurs simultaneously.

11. The method as recited in claim 9, wherein the step of administering the dopamine agonist and the step of administering the smoothened (Smo) agonist are separated by less than three hours.

12. The method as recited in claim 9, wherein the smoothened (Smo) agonist is purmorphamine (PUR).

13. The method as recited in claim 9, wherein the smoothened (Smo) agonist is a smoothened agonist (SAG).

14. The method as recited in claim 9, wherein the smoothened (Smo) agonist is administered in a dosage that is smaller than a dosage of the dopamine agonist.

15. The method as recited in claim 9, wherein the step of repeating changes dosage of the smoothened (Smo) agonist such that the dosage is larger than or equal to a dose of the smoothened (Smo) agonist given during the step of administering the smoothened (Smo) agonist.

16. A method for treating dyskinesia, the method comprising administering to a patient both amantadine and a smoothened (Smo) agonist.

17. The method as recited in claim 14, wherein the smoothened (Smo) agonist is purmorphamine (PUR).

18. The method as recited in claim 14, wherein the smoothened (Smo) agonist is a smoothened agonist (SAG).
